(19) United States
(12) Patent Application Publication
(10) Pub. No.: US 2007/0077552 A1
(43) Pub. Date: Apr. 5, 2007

(54) High throughput screen for inhibitors of polypeptide aggregation

(76) Inventors: Michael Hecht, Highland Park, NJ (US); Woo Jin Kim, Princeton, NJ (US); Christine Wurth, Loerrach (DE)

Correspondence Address: Connolly Bove Lodge & Hutz LLP;1007 North Orange Street, P.O. Box 2207, Wilmington, DE, 19899, US

(21) Appl. No.: 11/542,564

(22) Filed: Oct. 3, 2006

Related U.S. Application Data
(60) Provisional application No. 60/802,253, filed on May 19, 2006.
(60) Provisional application No. 60/723,597, filed on Oct. 4, 2005.

(51) Int. Cl. C12Q 1/04; C12Q 1/00

(52) U.S. Cl. 435/4; 435/34

(57) ABSTRACT

We have developed a high through-put screen capable of isolating inhibitors of polypeptide aggregation, such as Alzheimer's Disease polypeptide Aβ aggregation, or other disease state aggregating proteins, from amidst large libraries of candidate inhibitors. The screen uses a fusion of a polypeptide domain that self-aggregates, such as an Aβ42 domain characteristic of Alzheimer's disease plaques, to a reporter construct, such as Green Fluorescent Protein (GFP) or similar fluorescent protein. In the absence of inhibition, the rapid misfolding and aggregation of Aβ42 causes the entire fusion protein to misfold, thereby preventing fluorescence. Compounds that inhibit Aβ42 aggregation enable GFP to fold into its native structure, and can be identified by the resulting fluorescent signal.

### CROSS REFERENCE TO RELATED APPLICATIONS

This application asserts priority to US Provisional Application Nos. 60/723,597 filed Oct. 4, 2005; and 60/802,253 filed May 19, 2006, each of which is incorporated herein by reference in its entirety.

### BACKGROUND OF THE DISCLOSURE 1. Field of the Disclosure

This invention relates generally to assays for inhibitors of polypeptide aggregation, such as would be useful to treat diseases characterized by the formation of such aggregates. For example, the assays of the invention may be used to identify compounds to treat or prevent disorders such as Alzheimer's disease (AD), prion encephalopathies, Parkinson's disease and Huntington's disease. 2. Description of the Related Art

More than twenty human diseases are associated with the formation of deposits of aggregated proteins. Pharmaceutical agents directed to modulation of the aggregates are not generally available, however, in part because of the difficulty of screening for such agents.

Alzheimer's disease (AD) is one such disease that is characterized by the formation of protein aggregates. Aggregation of the Alzheimer's polypeptide, Aβ, is believed to play a causative role in the development of AD (1-7). The Aβ polypeptide aggregates are also referred to as a type of amyloid plaque. Amyloid plaques are extracellular deposits and are composed of amino acid chains arranged in a cross-beta pattern. Most drugs in clinical use for the treatment of AD target the symptoms of the disease, rather than its underlying molecular cause. Reducing the incidence, and slowing the progression, of diseases and syndromes associated with amyloid formation, such as AD, will require new drugs that disrupt the underlying molecular etiology of amyloid precursor aggregation. Therefore, compounds that inhibit production or aggregation of AP are attractive candidates as therapeutics for the prevention and treatment of AD.

Aβ polypeptides are produced in vivo by proteolytic cleavage of the amyloid precursor protein (APP) by β- and y-secretases (1). Because γ-secretase can cleave at several alternative sites, the resulting Aβ polypeptides vary in length. Forms of Aβ arise upon proteolytic cleavage by β- and/or γ-secretases, including Aβ(1-39), Aβ(1-40) known as Aβ40, Aβ(1-41), and Aβ(1-42) known as Aβ42. Aβ40 is produced in greater abundance; however, Aβ42 aggregates more readily and comprises the major component of amyloid plaque in diseased brains (8-10).

Although methods to screen for inhibitors of Aβ aggregation have been reported (11-12), these methods are hampered by several shortcomings. Published methods typically require synthetic Aβ polypeptide. Because Aβ42 aggregates during the synthetic procedure, synthesis of this 42-residue polypeptide is laborious and time-consuming. Consequently, synthetic Aβ42 is too expensive to use in screens aiming to analyze large libraries of compounds. In addition to its prohibitive cost, the aggregation of synthetic Aβ42 can also interfere with the efficacy of a screen: synthetic Aβ42 often contains oligomeric ‘seeds’ which can nucleate further aggregation. Since current models of AD pathogenesis implicate small oligomers on the pathway towards amyloid as the most toxic species (5, 6, 13-16), a screen relying on samples that contain pre-existing seeds may actually miss the most important inhibitors, including those that block the initial formation of soluble Aβ oligomers.

Some reported assays measure aggregation by turbidity or by fluorescence with Thioflavin T. However, turbidity is hard to quantify and is not useful for high through-put screening. Also, turbidity measurements and Thioflavin T fluorescence measurements are biased towards high order aggregates. Yet recent studies indicate that small aggregates may in fact be the more toxic species.

An object of the invention is to develop a high through-put screening method for inhibitors of polypeptide aggregation that does not rely on measurements of turbidity and that allows for identification of inhibitors of the early stages of aggregate formation.

The following US patents concern methods to screen for compounds to treat AD or methods or tools to identify useful treatments for AD: U.S. Patent No. 6,942,963 teaches methods for identifying treatments for neurotoxicity in AD caused by β-amyloid polypeptides; U.S. Patent No. 6,831,066 teaches modulators of β-amyloid polypeptide aggregation; U.S. Patent application publication No. 2005/0266502 is directed to methods for inhibiting β-amyloid protein production; U.S. Patent application publication No. 2003/0022151 is directed to screening methods for the identification of proteins and other molecules that cause the accumulation or stabilization of particular proteins; U.S. Patent No. 6,960,435 teaches a β-amyloid protein agglutination-controlling factor; U.S. Patent No. 6,867,018 is directed to AD secretase, amyloid polypeptide substrates for the secretase, and uses thereof; U.S. Patent application publication No. 2005/0138676 is directed to identification of genes involved in AD using Drosophila melanogaster; U.S. Patent application publication No. 2004/0024365 teaches fluorescent amyloid βP polypeptides and uses thereof; and U.S. Patent application publication No. 2005/0112720 is directed to complexes of β amyloid polypeptide prolyl isomerase chaperone and methods of making and using the chaperone.

### SUMMARY OF THE DISCLOSURE

The present invention is directed to screening assays for identifying substances that inhibit aggregation of a protein, such as proteins that aggregate in a disease state. In one embodiment, the invention is a screening assay for identifying inhibitors of polypeptide aggregation comprising:
a) forming a mixture of a test substance with an expression system, wherein the expression system comprises a nucleic acid encoding a fusion protein having a polypeptide domain that self-aggregates and a reporter protein domain that has an observable reporter function;
b) activating the expression system in the mixture such that the fusion protein is expressed;
c) monitoring the observable reporter function of the mixture having the test substance and comparing to the observable reporter function of the fusion protein in the absence of the test substance; and
d) determining from step (c) whether the test substance inhibits aggregation of the polypeptide domain; wherein step a) may be performed before, during, or after step b) and preferably step a) is performed before or simultaneously with step b).

In another embodiment, the invention comprises a method for assessing a structure/activity relationship for substances that inhibit polypeptide aggregation comprising:
a) identifying a first test substance and a structurally related second test substance, preferably wherein the test substances differ only in one chemical moiety,
b) forming a first mixture of the first test substance with an expression system, wherein the expression system comprises a nucleic acid encoding a fusion protein having a polypeptide domain that self-aggregates and a reporter protein domain that has an observable reporter function;
c) forming a second mixture of the second test substance with the expression system;
d) activating the expression system in the first mixture and the second mixture such that the fusion proteins in the first mixture and the second mixture are expressed;
e) monitoring the observable reporter function of the first mixture and comparing to the observable reporter function of the second mixture; and
f) determining from step (e) the relationship of structure to inhibition of polypeptide aggregation; wherein steps b) and c) may be performed before, during, or after step d).

### BRIEF DESCRIPTION OF THE FIGURES

The features and advantages of the assay will be more readily understood upon consideration of the following detailed description, taken in conjunction with the accompanying figures and illustrations. The figures may be viewed in color in the publication subsequent to the priority date of the present application: Kim et al., “A High-Throughput Screen for Compounds that Inhibit Aggregation of the Alzheimer's Peptide,” ACS Chem. Biol., 7: 461-469 (2006).

FIG. 1 illustrates a fluorescence-based screen using the Aβ42-linker-GFP fusion protein. In the absence of inhibition, the Aβ42 portion aggregates, causes the entire fusion protein to misfold and aggregate (left), and no fluorescence is observed. However, inhibition of Aβ42 aggregation enables GFP to form its native fluorescent structure (right). The illustration represents a properly folded GFP and a non-aggregated conformation of Aβ42. In the center is shown a triazine scaffold used to evaluate the screening assay. Combinatorial diversity was introduced at positions X, Y, and Z. A compound was added to each well, followed by E. coli cells capable of expressing the Aβ42-linker-GFP fusion protein. Positive controls had a fusion protein with the mutations F19S and L34P, which mutations are known to inhibit aggregation and enable fluorescence of the fusion protein. Negative controls contained no test compounds.

FIG. 2 illustrates, at the top, fluorescence digital results read-out in the format of a 96-well plate array. Compounds E2 (medium gray) and D2 (box) were chosen for further studies. The figure also illustrates the structure of D2 (middle) and E2 (bottom).

FIG. 3 illustrates the effect of graded amounts of compounds D2 and E2 on Thioflavin T fluorescence of synthetic Aβ42 polypeptide under a quiescent condition. Binding and fluorescence of Thioflavin T is a known assay for aggregates. E2 inhibited aggregation of synthetic Aβ42 polypeptide in a dose dependent manner. By comparison, compound D2 had little effect.

FIG. 4 illustrates the effect of graded amounts of compounds on Thioflavin T fluorescence of synthetic Aβ042 polypeptide incubated with agitation.

FIG. 5 illustrates electron microscopy of fibrils of Aβ42 after incubation with D2 or E2. Synthetic Aβ42 polypeptide was incubated for 5 days with various concentrations of either D2 or E2. At elevated concentrations, E2 inhibited fibrillogenesis. In contrast, the other test compound D2 was inactive at all concentrations.

FIG. 6 illustrates use of the assay for determination of structure/activity relationships (SAR) as described in example 4.

FIG. 7 illustrates the fluorescence observed with a fusion protein expressed in a cell-free assay. The fusion protein labeled Gm6 has a mutant Aβ protein having the F19S/L34P amino acid exchanges linked to a GFP. The fusion protein labeled wt has the wild type Aβ protein linked to a GFP. The linker and GFP are the same in the two fusion proteins.

FIG. 8 illustrates the fluorescence of Aβ42-linker-GFP in a cell-free assay in the absence or presence of tannic acid.

### DETAILED DESCRIPTION

As used herein, protein and polypeptide are synonymous.

Assay Methods

In the present assay methods, the solubility/aggregation behavior of a protein is coupled to a property that can be assayed on many samples in parallel. Such coupling is achieved by fusing the aggregating protein sequence to a reporter protein with an observable function. The reporter function is changed (either turned on or oft) by protein aggregation. Thus by monitoring the reporter protein function, one can readily determine the effect that a compound has on protein aggregation. In a preferred embodiment, the reporter function is fluorescence that is blocked by aggregation of the fusion protein but enabled by agents that inhibit protein aggregation.

The compound to be assayed is contacted with an expression system that comprises a nucleic acid, preferably DNA, encoding the fusion protein. The user activates expression of the fusion protein. In this manner, activation of the expression of the protein can be timed to occur after the test compound is in contact with the expression system and thus the compound can be observed for its effects on the earliest formation of aggregation from the point of initial protein expression onwards.

The assay can be performed using any expression system that allows for the expression of the fusion protein in a mixture with a test compound. By the term expression system is meant a system that comprises a nucleic acid and the necessary biological and/or chemical elements to allow for transcription and translation of that nucleic acid. This includes a recombinant cell line, such as an E. coli cell, in a cell culture that has the materials to allow for protein expression, and also includes a cell-free transcription/translation system such as are known in the art for protein expression without a cell. Cell-free systems are commercially available from Promega Corp. and Roche Applied Science, a division of Roche Diagnostics Corporation, among other suppliers. The recombinant cell line suitable for the assay is not limited to E. coli and can include any bacterial, fungal, archaeal, plant, or animal cell, or indeed any living cell having a controllable nucleic acid encoding a fusion protein.

The expression system may be one in which expression of a nucleic acid can be activated, or deactivated, by the user. For example, the process of activating the controllable source can be by use of isopropyl-β-D-thiogalactopyranoside (IPTG). Expression may also be initiated by changing the media composition or the temperature using initiation means known in the art. Alternatively, the assays may also be used with an expression system that is not readily activated by the user.

The fusion protein has a reporter protein domain. By the term “reporter protein domain” is meant a protein (or protein domain) that has a function that is readily observed or measured. By the term “observable reporter function” is meant a function of the reporter protein that is readily observed or measured, such as fluorescence. The reporter protein domain in the fusion protein preferably comprises a protein domain capable of exhibiting an observable property upon correct folding, such as fluorescing upon correct folding. By the term “fluorescent protein” is meant a polypeptide that, in response to incident radiation in the visible or ultraviolet spectra, emits radiation at a wavelength longer than the incident radiation. The term “fluorescent domain” is used to indicate the portion of a fluorescent protein having a structure distinct from adjacent portion(s) of the protein and which is responsible for the fluorescence. In practice, fluorescent proteins and fluorescent protein domains generally emit in the visible portion of the spectrum. Suitable reporter protein domains include “green fluorescent protein” (GFP), GFP-like proteins, and proteins which require a co-factor to fluoresce, such as luciferase.

Suitable GFP proteins include the GFP domains used in the fusions reported by Waldo et al. (17) in studies identifying proteins having productive folding. Waldo et al. (17) was concerned with problems of protein misfolding and aggregation during recombinant expression. Suitable GFP proteins are also disclosed in Wurth et al. (18). In that work, GFP fusion constructs with mutations in Aβ42 were used to search for the sequence determinants of Aβamyloidogenesis. Kim et al. (19) also disclose suitable GFP in an article reporting further mutagenesis experiments of the Aβpolypeptide. Another GFP fusion construct was reported by Coustou-Linares et al. (20). Coustou-Linares et al. discloses in vivo aggregation of the HET-s prion protein of the fungus Podospora anserina.

Wild type GFP from the jellyfish Aequorea victoria has a major excitation peak at a wavelength of 395 nm and a minor one at 475 nm. The fluorescence emission peak of GFP is at 509 nm which is in the lower green portion of the visible spectrum, hence the name. The GFP-like protein from the sea pansy (Renilla reniformis) has a single major excitation peak at 498 nm. Modified forms of GFP can also be used. Modified GFPs include mutants with increased fluorescence and/or in which the major excitation peak has been shifted, for example, to 490 nm with the peak emission kept at 509 nm. Modified GFPs can also include mutants with a shifted emission peak. Thus, color mutants are suitable, including the cyan fluorescent protein and the yellow fluorescent protein. The term “mutant form of GFP” as used herein denotes fluorescent proteins that are GFP-like including, without limitation, R. reniformis GFP-like protein, synthetic mutants of wild-type GFP, modified GFP, and color mutants, in that the properly folded proteins have generally similar fluorescence excitation/emission spectra and misfolded proteins are substantially not fluorescent.

The fusion protein preferably has a linker attaching the polypeptide domain that self-aggregates to the reporter protein domain. The linker used in the fusion protein is such that the folding and aggregation state of the aggregate-forming polypeptide domain is tied to the activity of the reporter protein domain. For example, the linker is such that aggregation caused by the aggregating protein domain affects the folding activity required for fluorescence of the reporter protein domain. Such a linker can be any reasonable length, such as 5-30 amino acids in length. The linker should not be too long or too flexible as this may allow for some dissociation of activity between the protein aggregation and the folding of the fluorescent protein domain. In one embodiment of the fusion construct, Aβ42 is separated from the N-terminus of GFP by a linker encoding the sequence: Gly-Ser-Ala-Gly-Ser-Ala-Ala-Gly-Ser-Gly-Glu-Phe (SEQ ID NO:1). This sequence was shown previously to be effective in coupling the aggregation state of N-terminal fusions with the fluorescence of GFP (17-19). Another suitable linker is (Gly-Gly-Gly-Ser)₄ (SEQ ID NO: 2) shown to be useful in GFP fusions previously (17). Longer or more disordered linkers are less favored because they could uncouple the aggregating properties of the self-aggregating polypeptide domain from the observable reporter function.

The fusion protein also has a polypeptide domain that self-aggregates. By the term “polypeptide (or polypeptide domain) that self-aggregates” is meant any protein that forms aggregates with other of the same proteins. Preferably, the polypeptide domain that self-aggregates is a polypeptide characteristically found in aggregates in a disease state and for which the aggregation is believed to play a causative role in the symptoms or progression of the disease. Such self-aggregating polypeptides are found in diseases such as AD (β-amyloid), Parkinson's, Huntington's disease, Creutzfeldt-Jacob Syndrome and other prion diseases, or type 2 diabetes mellitus (amylin). The term also encompasses fragments of such polypeptides and analogs thereof. These diseases involve formation of protein deposits in or near cells, sometimes bound to basement membrane proteins, which may have glycosaminoglycans.

Suitable substances for testing are substances that have a size and solubility such that they would be expected to be able to interact with the fusion protein in the environment of the expression system. For example, the test substance may be a small molecule having a molecular weight less than about 2000 Daltons, an oligopeptide, or a non-peptide small molecule, meaning a molecule having a molecular weight less than about 2000 Daltons and not consisting of L-amino acid residues.

The inventive assays may be used in a high through-put screen. High through-put screening is a process in which libraries of compounds are tested for biological activity against target molecules such as the aggregating protein domains of the fusion proteins described herein. High through-put screening seeks to screen large numbers of compounds rapidly and in parallel. For example, using microtiterplates and automated assay equipment, a pharmaceutical company may perform as many as 100,000 assays per day in parallel. The assays of the present invention are well designed for use in high through-put screening because the reporter protein domain can be chosen such that it exerts a property that is readily measurable by a mechanical device and the data stored for subsequent analysis. For example, fluorescent reporter domains such as GFP are desirable in high through-put screening.

The assays of the invention may involve more than one measurement of the observable reporter function. Multiple measurements may allow for following the biological activity over incubation time with the test substance. In one embodiment, the fluorescence of the mixture is measured at a plurality of times to allow monitoring of the effects of the test compound at different incubation times.

In another aspect, the invention provides for following the screening assay described above with a subsequent assay to further identify whether the identified inhibitor has properties desirable for the intended use. For example, the screening assay may be followed by a second assay selected from the group consisting of measurement of any of: the kinetics of aggregation, sedimentation of aggregates, binding of a dye to an aggregate, chemical cross-linking, electrophoresis, fibril morphology, turbidity, bioavailability, toxicity, or pharmacokinetics, but is not limited to these methods.

It is envisioned that the assay of the invention can also be performed by co-transfecting the recombinant cells with a nucleic acid encoding the fusion protein discussed above and with nucleic acid encoding a random sequence of peptides. In this manner, a random polypeptide library can be assayed for inhibitory activity against an aggregating protein of interest.

In another embodiment, the invention is a method for assessing a structure/activity relationship for substances that inhibit polypeptide aggregation by using the fusion protein and the expression system discussed above. In the method for assessing structure/activity relation, preferably the substances subjected to the assay are similar in chemical structure except for a variation at one substituent on a common core structure. By comparing the relative inhibition strengths of molecules having such similar structure, the structure/activity relationship can be determined.

Diseases Characterized by Aggregation of Protein

The method of the invention is useful for identifying inhibitors of polypeptide aggregation. The assay methods may be used to measure amyloid aggregation for any type of amyloid by appropriately modifying the nucleic acid encoding the fusion protein. Amyloids have been categorized by the type of amyloidogenic protein contained within the amyloid. Non-limiting examples of amyloids which can be used in the fusion proteins of the assay, as identified by their amyloidogenic protein, include: β-amyloid (Aβ), amyloid A, amyloid κ L-chain or amyloid λ L-chain of immunoglobulin, amyloidogenic ,ββ-microglobulin (Aβ2M), amyloidogenic transthyretin (ATTR), islet amyloid polypeptide (IAPP or amylin), atrial naturetic factor, procalcitonin, gelsolin, cystatin C, amyloidogenic apolipoprotein A-I (AApoA-I), amyloidogenic apolipoprotein A-II (AApoA-II), fibrinogen-associated amyloid, lysozyme-associated amyloid, Huntingtin protein, Parkinson's Disease protein parkin, polyglutamine repeats, and AScr or PrP-27.

β-Amyloid is associated with AD, Down's syndrome, and hereditary cerebral hemorrhage amyloidosis. When the assay is used to screen for substances to treat or prevent AD, Aβ42 is preferred to Aβ40 as the aggregating polypeptide domain of the screen because the longer polypeptide is the major component of senile plaque. Also, the ratio of Aβ42/Aβ40 is increased in diseased brains (8, 9). The 42-residue polypeptide also forms aggregates more rapidly in vitro (10). However, any a β-amyloid polypeptide may be used in the fusion protein of the inventive assay to identify compounds for the prevention or treatment of AD.

Amyloid A is associated with reactive amyloidosis, familial Mediterranean fever, and familial amyloid nephropathy with urticaria and deafness. Amyloid κ L-chain or amyloid λ L-chain are associated with idiopathic, myeloma or macroglobulinemia. Aβ2M is associated with chronic hemodialysis. ATTR is associated with familial amyloid polyneuropathy, familial amyloid cardiomyopathy, isolated cardiac amyloid, and systemic senile amyloidosis. IAPP or amylin is associated with type 2 diabetes. Atrial naturetic factor is associated with isolated atrial amyloid. Procalcitonin is associated with medullary carcinoma of the thyroid. Gelsolin is associated with familial amyloidosis of the Finnish type. Cystatin C is associated with hereditary cerebral hemorrhage with amyloidosis. AApoA-I is associated with familial amyloidotic polyneuropathy of the Iowa type. AApoA-II is associated with accelerated senescence in mice. AScr or PrP such as PrP-27 is associated with the prion diseases: Scrapie in sheep, Creutzfeldt-Jacob disease in humans, variant Creutzfeldt-Jacob disease in humans, Gerstmann-Straussler-Scheinker syndrome in humans, fatal familial insomnia in humans, and bovine spongiform encephalitis. The prion diseases include transmissible spongiform encephalopathies and chronic wasting diseases. Huntingtin protein is associated with Huntington's Disease. Parkinson's Disease is associated with parkin, and possibly with DJ-1. The inventive assay methods may be used to identify compounds to treat or prevent any of these diseases by appropriate modification of the fusion protein to express the aggregating protein.

Screening Methods for Substances to Treat AD

A high throughput screen for inhibitors of Aβ aggregation requires the solubility/aggregation behavior of Aβ to be coupled to a property that can be assayed on many samples in parallel. Such coupling can be achieved by fusing the Aβ sequence to a reporter protein with an observable function that is blocked by Aβ aggregation, but enabled by agents that inhibit Aβ aggregation.

In one embodiment, the screen achieves this goal by fusing the sequence of Aβ42 to Green Fluorescent Protein (GFP), such as by preparing a nucleic acid construct having the relevant two coding regions. The fluorescence of the Aβ42-GFP fusion protein depends on the folding and solubility of the fused Aβ42. It is believed that the slow rate of folding of GFP into its native fluorescent structure (21) may assist in the assay. Misfolding and aggregation of the Aβ42 sequence causes the entire Aβ42-GFP fusion protein to misfold prior to formation of the correct fluorescent structure. Inhibitors that retard (or block) Aβ42 aggregation enable GFP to fold into its native structure, and can be distinguished by the resulting fluorescent signal. In a preferred embodiment, the fusion protein consists essentially of an amyloid Aβ42 domain covalently linked by a polypeptide linker to a GFP domain.

In traditional screens relying on turbidity or binding of thioflavin T, a compound is scored as a hit if it prevents assembly into amyloid fibrils. Since fibrils occur late in the aggregation pathway, a potential disadvantage of these older screens is the likelihood that some compounds isolated by these screens will inhibit the later steps of amyloidogenesis, but fail to inhibit the upstream formation of toxic soluble oligomers. The Aβ42-GFP screen for misfolding and aggregation disclosed here is believed to identify compounds that block early misfolding and aggregation without requiring the formation of amyloid fibrils.

Fusions of wild-type Aβ42 to GFP do not fluoresce, and expression of the Aβ42-linker-GFP fusion protein in E. coli yields non-fluorescent colonies (18, 19). These fusions form an artificial genetic system in E. coli useful to screen for mutations in Aβ42 that inhibit aggregation (18). Non-aggregating mutants were isolated by screening random mutations in Aβ42 for those that produce green fluorescent colonies. The ability of the selected amino acid substitutions to diminish the aggregation of Aβ42 was confirmed by biophysical studies of mutant versions of the synthetic 42-residue polypeptide.

Sensitivity of the Screen for Inhibitors of Aβ Aggregation

An effective screen must be sensitive enough to detect compounds with relatively low levels of inhibitory activity. This is important for two reasons: First, initial implementation of a screen typically searches for lead compounds, rather than final drugs. Therefore, a screen should be sensitive enough to detect first-generation compounds with only moderate effects on aggregation. (Such leads can be optimized at later stages.) Second, detection of compounds with low activity is important because drugs with modest effects on aggregation may in fact be sufficient to treat AD: In early onset AD caused by familial mutations in APP or in the presenilins, levels of Aβ42 are increased by as little as 30% (3). This small increase in Aβ42 can advance the onset of AD by 30-40 years. Therefore, compounds with only moderate inhibitory activity may suffice to delay the onset of AD to the point where it is no longer a major health problem. The Aβ42-linker-GFP fusion system described here has the required level of sensitivity, as shown by earlier work using the fusion protein to screen for mutations in Aβ42 that diminish aggregation (19).

The Aβ42-linker-GFP fusion system is sensitive to inhibitory effects at sites throughout the length of the 42-residue Aβ sequence (18). The presence of a linker following residue 42 does not interfere with inhibitory effects on the C-terminal residues of Aβ42, which are known to be important for aggregation (8-10, 23), because the Aβ42-linker-GFP fusion protein can discriminate small differences in aggregation rate caused by mutations throughout Aβ42, including those at residues 41 and 42 (18, 19).

When screening for compounds that inhibit aggregation, it is important to ensure that the screen does not inadvertently identify generic inhibitors of protein folding. This possibility must be considered because aggregation into β-sheet fibrils and folding into native globular structures are similar processes: Both involve self-assembly of a polypeptide into an ordered structure. Although Aβ42 aggregation is intermolecular and protein folding is intramolecular, the two processes are governed by the same types of interactions (hydrogen bonding, the hydrophobic effect, propensities for secondary structure, side chain packing, etc.). Therefore, a screen for inhibitors of aggregation should not inadvertently identify inhibitors of protein folding, particularly the folding of β-sheet proteins. The Aβ42-linker-GFP fusion system is internally controlled for this possibility. A positive signal (fluorescence) is required to identify hits; and this signal is observed if and only if GFP folds into its native structure. Therefore, generic inhibitors of protein folding will not be isolated by this screen. Moreover, since GFP is a β-sheet protein, generic inhibitors of β-sheet structure will not be isolated. These undesirable effects are ‘weeded out’ by the requirement for correct folding of GFP.

Oligomers of Aβ

While insoluble amyloid plaque has long been thought to play a causative role in AD, recent work suggests that smaller aggregates (Aβ oligomers) on the pathway towards amyloid may in fact be more toxic than insoluble plaque (5, 6, 13-16, 23, 24). It is important to consider what stages of aggregation might be blocked by compounds scored as ‘hits’ in high throughput screens. The exact level of Aβ42 aggregation (e.g., dimers, tetramers, or hexamers) that prevents fluorescence of the Aβ42-linker-GFP fusion protein is not yet known. Without being limited to a particular mechanism, it seems likely that the non-fluorescent phenotype of the misfolded aggregate would be apparent at or before the dodecameric stage, which has been proposed to be the toxic species responsible for memory impairment in AD (16). Once active inhibitors are isolated, the exact oligomerization stage at which they function and the precise mechanism of their action can be assessed by biophysical studies.

For a screen to find inhibitors of the earliest stages of aggregation, it is important that the compounds being tested are present prior to the initial steps of the aggregation pathway. For screens that relied on synthetic Aβ42 polypeptide, this posed a serious challenge: Because Aβ42 aggregates so readily, it is difficult to prepare aqueous samples that are entirely free of partially aggregated seeds. The presence of these seeds (which presumably contain oligomers) meant that the species that must be inhibited would have already been present prior to addition of putative inhibitors. Consequently, screens using synthetic Aβ42 polypeptide could miss the very compounds that ultimately will provide leads for the development of anti-AD therapeutics. The Aβ42-linker-GFP fusion system overcomes these problems: In one embodiment of the new screen, Aβ42 is not present prior to addition of the test compounds; expression of the Aβ42-linker-GFP fusion protein is induced only after the test compounds have been added.

Applications of the Screen to AD

Certain classes of compounds that successfully inhibit Aβ42 aggregation may nonetheless be missed by the screen. Two examples include compounds that fluoresce at the same wavelength as GFP, and compounds that are toxic to cells. To enable screening of libraries containing green fluorescent compounds, it may be necessary to use a mutant form of GFP that fluoresces in another part of the spectrum, e.g. yellow fluorescent protein (25, 26). Cytotoxic compounds will also be missed by our screen; however, this may be advantageous since such compounds are unlikely to be suitable as drugs.

The version of the Aβ42-GFP fluorescent screen described above relies on expression of the fusion protein in E. coli, or other cell. Screening in a cell, e.g. E. coli, has several advantages: It is fast, inexpensive, and highly reproducible. Moreover, it favors compounds that (i) are non-toxic and (ii) readily penetrate biological barriers. Nonetheless, expression in E. coli may also introduce a limitation: To be scored as a hit in this cell-based screen, a compound must enter the bacterial cell. Inhibitors of Aβ aggregation that fail to enter cells will not produce fluorescent signals, and will escape detection in this initial version of our screen. The significance of this limitation will depend on the type of library being screened. Some chemical moieties are inherently more likely than others to enter cells (27).

Another embodiment of the disclosure is directed to an Aβ42-GFP fluorescent screen in which the fusion protein is expressed in vitro using a cell-free transcription and translation system. This cell-free system readily distinguishes between aggregating and non-aggregating mutants of Aβ42.

### EXAMPLES

### Example 1

Fluorescent Screen for Inhibitors of Aβ Aggregation

To generate a positive control, random mutagenesis was carried out on the Aβ42 sequence. A library of mutagenized sequences was transformed into E. coli and plated. Fusion proteins containing wild-type Aβ42 produce colorless colonies. Mutations in Aβ42 that inhibit aggregation yield green fluorescent colonies. Green colonies and some colorless controls were picked and cultured in liquid medium. Cultures expressing a fusion protein with the mutations Phe19Ser, Leu34Pro in Aβ42 are green, because these mutations inhibit Aβ42 aggregation, enabling GFP to fold and be capable of fluorescence (18). This mutant is thus suitable for use as a positive control.

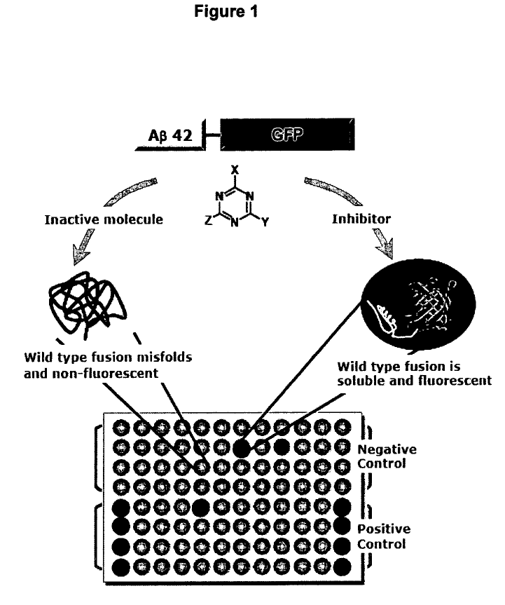

The vector for expressing the Aβ42-linker-GFP fusion protein was described previously (18, 19) and the contents of these references are specifically incorporated by reference herein for all purposes. The mutant GFP which was used was described by Waldo et al. (17) and was based in part on a mutant GFP described by Crameri et al. (28). The mutant GFP is a soluble variant that was engineered based on a variant that folds well in E. coli using site-directed mutation to eliminate internal NdeII and BAMHI sites and incorporate the red-shift S65T mutation and the folding mutation F64L (28). Strain BL21(DE3) E. coli cells (29) harboring the Aβ42-linker-GFP fusion protein vector were grown in LB media supplemented with 35 μg ml⁻¹ kanamycin. When cultures reached an OD600 of about 0.8, 100 μL of culture was transferred to each well of 96-well plates. Candidate compounds from a triazine library were added to each well, and protein expression was induced by adding IPTG to a final concentration of 1 mM. Samples were incubated with gentle agitation at 37° C. Following 3 hours of incubation, the fluorescence of each well was measured at 512 nm (excitation 490 nm) using an automated plate reader (SpectraMAX Gemini XS, Molecular Devices). To verify that cell densities were consistent across all samples, the OD600 was also measured. Compounds were tested in quadruplicate: twice at a final concentration of 30 μM, and twice at a final concentration of 100 μM. Several wells containing specific triazine derivatives fluoresced at levels significantly above background (FIG. 1). The fluorescent hits are considered as putative inhibitors of Aβ42 aggregation. The identification of hits was consistent across several repetitions.

The screen was tested on a library of approximately 1000 compounds based on a triazine scaffold. The triazine-based library was obtained from Dr. Y-T Chang at NYU (30, 31). This library was prepared by varying the substituents at positions X, Y, and Z on the scaffold shown in FIG. 1. The substituents at positions X, Y, and Z are described in references 30 and 31.

The triazine library described above was used as a pilot library to demonstrate that the Aβ42-GFP screen can indeed distinguish hits from inactive compounds. Although triazines may not be the optimal scaffold for drug discovery, we note that compound RS-0466, which was shown by Selkoe and coworkers to block Aβ oligomerization and rescue long-term potentiation, is a triazine derivative (38).

Overall, screening a library of approximately 1,000 compounds required several hours. Scale-up procedures using robotic sample handling will enable screening of much larger libraries on a high throughput scale.

In the absence of inhibition, the Aβ42 portion of the fusion protein aggregates rapidly and causes the entire Aβ42-linker-GFP fusion protein to misfold and aggregate (see FIG. 1, left). Therefore, no fluorescence is observed. However, inhibition of Aβ42 aggregation enables GFP to form its native green fluorescent structure (FIG. 1, right). (The medium gray part of the ribbon diagram shows the structure of GFP; the light gray part is merely a schematic representation of a nonaggregated form of Aβ42). The triazine scaffold is shown at the center of the figure. A 96-well plate is shown at the bottom of the figure. Compounds were added to each well, followed by E. coli cells expressing the Aβ42-linker-GFP fusion protein. Negative (colorless, shown as light gray) and positive (green, shown as medium gray) controls are shown in the columns on the edges of the plate. For negative controls, no test compounds were added to the wells. For positive controls, the wild type Aβ42-linker-GFP fusion protein vector was replaced with a fusion protein vector in which the Aβ42 sequence contained mutations F19S and L34P (Gm6).

Results of the Fluorescent Screening Assay

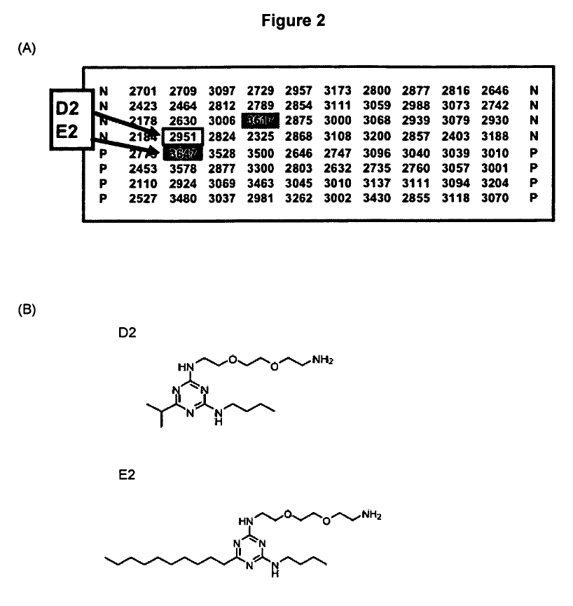

Screening results for the triazine library are illustrated in FIG. 2. (A) shows a digital readout of the fluorescence of E. coli cells expressing the Aβ42-linker-GFP fusion protein in the presence of compounds from a combinatorial library of triazine derivatives. ‘N’ denotes negative control wells without compound (mean: 2706, standard deviation: 238). ‘P’ denotes positive control wells expressing a GFP fusion protein to the soluble F19S/L34P mutant of Aβ42 (mean: 4610, standard deviation: 155). Compounds that reproducibly yielded florescence signals 3 standard deviations above the mean of the negative control were highlighted in medium gray. Compounds E2 (gray) and D2 (control) were chosen for further studies. (B) shows structures of the aggregation inhibitor, E2 and the inactive compound, D2.

### Example 2

Thioflavin T Studies Confirm the Activitv of a Selected Inhibitor

The Aβ42-linker-GFP fluorescence screen was successful at identifying potential inhibitors of aggregation. Because the screen relies on several artificial features, it is worth considering whether compounds isolated by this screen actually inhibit aggregation of the Aβ42 polypeptide in a well-defined biochemical system. The artificial features of the Aβ42-linker-GFP screen include (i) a fusion protein in which the relevant 42-residue Aβ sequence is only a small fraction of the 292-residue fusion protein, and (ii) expression in E. coli, which is clearly not the natural system for Alzheimer's disease. Consequently, we performed an experiment to verify that fluorescence observed for the Aβ42-linker-GFP fusion protein expressed in E. coli indeed correlates with diminished aggregation of the Aβ42 polypeptide.

Mutations in Aβ42 that yield green fluorescence in the context of the Aβ42-linker-GFP fusion protein expressed in E. coli diminish the aggregation of synthetic Aβ42 polypeptide studied in vitro (18). The effects of the triazine derivatives E2 and D2 on the aggregation behavior of synthetic Aβ42 polypeptide was measured to establish the correlation between the aggregation of Aβ42 in vitro and generation of a fluorescence signal in the screening assay.

Soluble monomeric Aβ42 polypeptide can be prepared using organic solvents, sonication, and filtration (32). When such samples are diluted into aqueous buffer, the polypeptide aggregates into fibrillar amyloid structures, which can be assayed by the binding and resulting fluorescence of Thioflavin T (33). The rate of Aβ42 aggregation depends on the conditions of the incubation: Under ‘quiescent’ conditions, aggregation is slow, whereas agitation causes Aβ42 to aggregate more rapidly.

Synthetic Aβ42 polypeptide (34) was incubated at 20 μM in Phosphate Buffered Saline (PBS, 50 mM NaH₂ PO₄, 100 mM NaCI, 0.02% NaN₃) in the presence or absence of candidate inhibitors at various concentrations. Following incubation with or without agitation, Thioflavin T was added to a final concentration of 7 μM, and fluorescence was measured at 490 nm (excitation 450 nm).

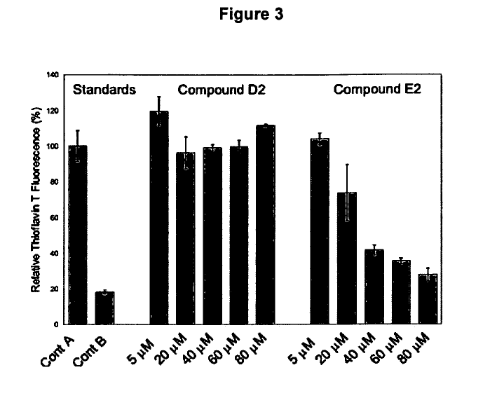

The effects of compounds D2 and E2 on the aggregation of Aβ42 were evaluated under both quiescent and agitated conditions. For the quiescent conditions, synthetic Aβ42 at a concentration of 20 μM was incubated for 2 hours in the presence of various concentrations of either D2 or E2. Fibril formation was assayed by the shifted fluorescence of Thioflavin T that accompanies binding to fibrils (34). As shown in FIG. 3, compound E2 inhibits aggregation in a concentration dependent manner, with an IC₅₀ around 30 μM. At 80 μM, E2 produces nearly complete inhibition of Aβ42 aggregation. In contrast, D2 shows no inhibitory effect. Control A is synthetic Aβ42 alone. Control B is buffer alone. Additional controls showed that in the absence of polypeptide, compounds D2 and E2 had no effect on Thioflavin T fluorescence (data not shown). Fluorescence is shown as a percentage of the control (synthetic Aβ42 alone).

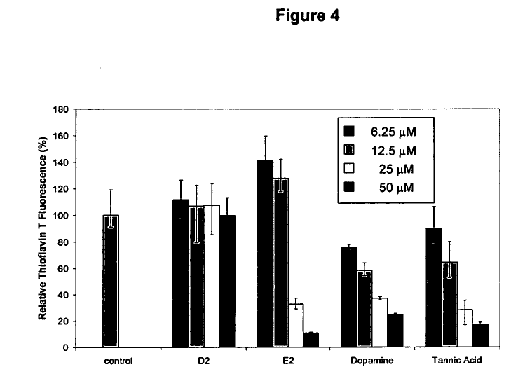

Compounds D2 and E2 were also tested for their inhibitory effects under agitated incubation conditions. Here the effect was even more dramatic: While the control compound D2 was inactive, the selected compound E2 caused a 90% reduction in Thioflavin T fluorescence at a concentration of only 50 μM (FIG. 4). The inhibitory effect of E2 was compared to dopamine and tannic acid, which were shown previously to inhibit A42 aggregation (35, 36). As shown in FIG. 4, at concentrations of 25 and 50 μM, the inhibitory effect of E2 was similar to, or slightly better than, dopamine or tannic acid.

### Example 3

Electron Microscopy

A42 polypeptide at a concentration of 20 μM in PBS buffer was incubated in the presence or absence of the test compounds at various concentrations. Following 5 days of incubation at 37° C. under quiescent condition, Formvar carbon-coated grids were floated on a drop of the sample for 2 min. The grids were blotted using filter paper and then stained for 2 min with freshly made 1% uranyl acetate. Samples were imaged using a Zeiss 912ab Electron Microscope.

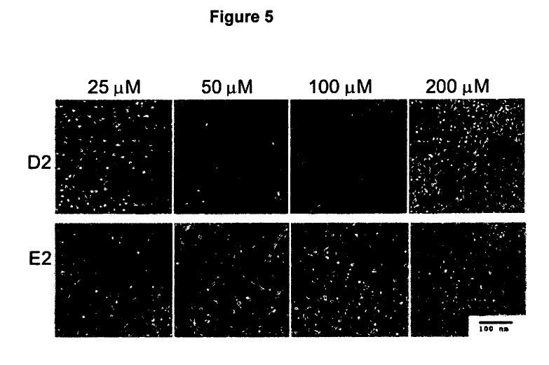

The ability of E2 to inhibit the assembly of Aβ42 into amyloid fibrils was also assessed by electron microscopy (EM). Aβ42 polypeptide was incubated for five days, either alone or in the presence of compounds D2 or E2. Five days is a relatively long incubation time; in the absence of inhibitors, Aβ42 readily forms visible fibrils after one or two days (data not shown). Following the 5-day incubation, samples were stained with uranyl acetate and imaged by EM. As shown in FIG. 5, the control compound D2 was inactive at all concentrations. Compound E2, however, inhibited fibrillogenesis in a dose dependant manner. At 50 μM, E2 had no effect, at 100 μM only short fibrils (perhaps ‘protofibrils’) were observed, while at a concentration of 200 μM, compound E2 completely inhibited fibril formation.

The results shown in FIGS. 3-5 confirm that the novel fluorescence-based assay using an Aβ42-linker-GFP fusion protein expressed in E. coli can detect compounds that indeed inhibit aggregation and/or amyloidogenesis of the Aβ42 polypeptide.

### Example 4

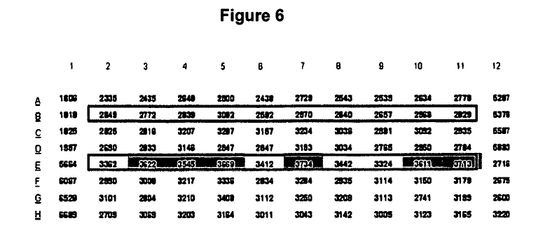

Assessing Structure/Activity Relationships Because the Aβ42-linker-GFP fusion protein is sensitive enough to detect both low and high levels of inhibitory activity, the screen can be used to determine structure/activity relationships (SAR). For example, as shown in FIG. 2, compounds D2 and E2 are identical at positions X and Y, but differ at position Z. Screening collections of molecules that differ at only one position should establish the relationship between substituents at each position and the resulting level of inhibition. Thus, in FIG. 6, which shows the digital readout of a 96-well plate testing a series of compounds, in any given row, the substituents at positions Y and Z were held constant, while the functional group at position X was varied. As shown in the figure, most compounds in row E produced a positive signal, indicating that variation of the chemical moieties at position X has little effect on activity. In contrast, compounds in other rows (e.g. row B) and on other plates (data not shown) were inactive, indicating that the selected functional groups at positions Y and Z are important for inhibitory activity.

### Example 5

A Cell-Free Screening Assay

In one embodiment, the assay uses a vector encoding Aβ42-linker-GFP in a cell-free in vitro transcription and translation system. For example, the basic protocol of the cell-free assay can involve the following steps: 1) Culture 500 ml to 1L of E. coli X1Blue strain harboring Aβ-linker-GFP fusion vector until the density of the suspension reaches O.D 0.9; 2) At O.D 0.9, spin down the cells at 4,000 g for 15 min; 3) Extract the vector using the Qiagen plasmid mega kit. (Cat. No. 12183); 4) After extraction, remove contaminants using phenol extraction; 5) Add E. coli T7 S30 extract system for circular DNA (Promega, Cat. No. L1130) into each well of a 96-well plate; 6) Add control or test substances (e.g. putative drug molecules or vectors that express inhibitory polypeptide) to each well; 7) Add Aβ-linker-GFP fusion vector, and express at RT or at 37 ° C. for 3 hrs; and 8) Measure the fluorescence emission at 510 nm (using an excitation at 490 nm). One of skill in the art will recognize that other transcription/translation systems, or a modified system, can be used. Likewise, the details of the protocol can be varied and optimized to accommodate other amyloidogenic proteins, to enhance the relative fluorescence, to accommodate the needs of the assayist, or a combination thereof.

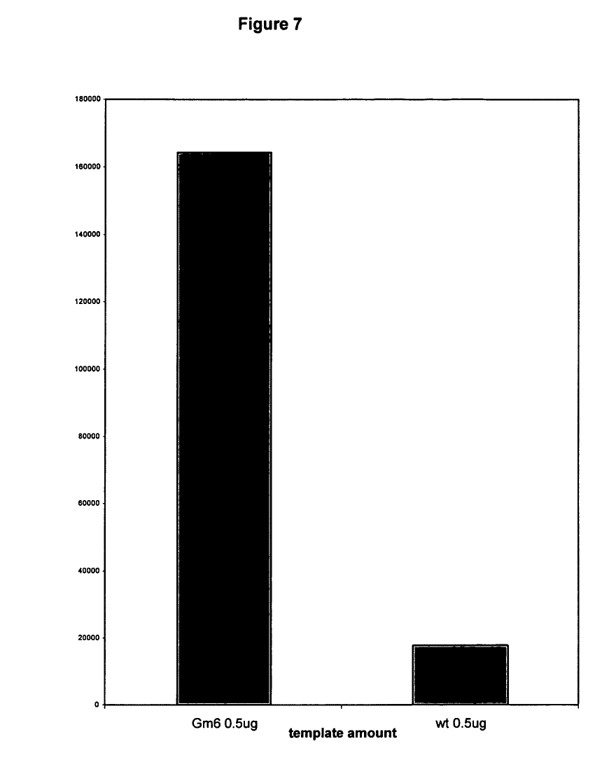

FIG. 7 shows a comparison of the fusion protein having a mutant Aβ protein (the soluble F19S/L34P mutant, at 0.5 μg) and the fusion protein having the wild type Aβ protein using the cell-free assay. The mutant Aβ fusion protein shows about eight-fold the fluorescence of the wild type fusion protein, consistent with proper folding of GFP, and lack of aggregation of Aβ, in the mutant.

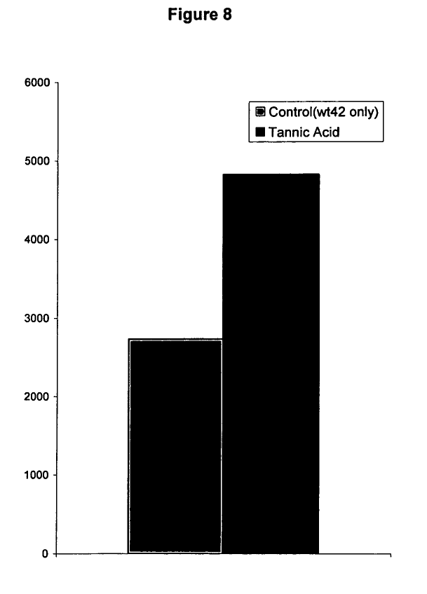

FIG. 8 illustrates the inhibition of fluorescence of Aβ42-linker-GFP in a cell free system. Wild-type Aβ42-linker-GFP was expressed in the absence (gray) or presence (black) of 10 μM tannic acid. The enhanced fluorescence indicates that tannic acid partially inhibits aggregation of the Aβ42-linker-GFP fusion protein in the cell-free system.

Although the above disclosure has been directed to several aspects of a method of assaying inhibitors of protein folding and/or aggregation, the method of the present disclosure is not limited to such an implementation.

It will be obvious that the present methods may be varied in many ways. Such variations are not to be regarded as a departure from the spirit and scope of the methods, and all such modifications as would be obvious to one skilled in the art are intended to be included within the scope of the following claims. The breadth and scope of the present invention is therefore limited only by the scope of the appended claims and their equivalents. All of the references and patent publications referred to herein are incorporated herein by reference in their entireties.

### REFERENCES

(1) Selkoe, D. J. (2001) Alzheimer's disease: genes, proteins, and therapy. Physiol. Rev. 81, 741-766.

(2) Koo, E., Lansbury, P. J., and Kelly, J. (1999) Amyloid diseases: abnormal protein aggregation in neurodegeneration. Proc. Natl. Acad. Sci. USA 96, 9989-90.

(3) Scheuner, D., Eckman, C., Jensen, M., Song, X., Citron, M., Suzuki, N., Bird, T. D., Hardy, J., Hutton, M., Kukull, W., Larson, E., Levy-Lahad, E., Viitanen, M., Peskind, E., Poorkaj, P., Schellenberg, G., Tanzi, R., Wasco, W., Lannfelt, L., Selkoe, D., and Younkin, S. (1996) Secreted amyloid-β protein similar to that in the senile plaques of Alzheimer's disease is increased in vivo by the presenilin 1 and 2 and APP mutations linked to familial Alzheimer's disease. Nat. Med. 2, 864-70.

(4) Geula, C., Wu, C., Saroff, D., Lorenzo, A., Yuan, M., and Yankner, B. (1998) Aging renders the brain vulnerable to amyloid-β protein neurotoxicity. Nat. Med. 4, 827-31.

(5) Walsh, D. M., Klyubin, I., Fadeeva, J. V., Cullen, W. K., Anwyl, R., Wolfe, M. S., Rowan, M. J., and Selkoe, D. J. (2002) Naturally secreted oligomers of amyloid-β protein potently inhibit hippocampal long-term potentiation in vivo. Nature 416, 535-9.

(6) Walsh, D. M., Klyubin, I., Fadeeva, J. V., Rowan, M. J., and Selkoe, D. (2002) Amyloid-β oligomers: Their production, toxicity and therapeutic inhibition. Biochem. Soc. Trans. 30, 552-557.

(7) Irie, K., Murakami, K., Masuda, Y., Morimoto, A., Ohigashi, H., Ohashi, R., Takegoshi, K., Nagao, M., Shimizu, T., and Shirasawa, T. (2005) Structure of β-amyloid Fibrils and Its Relevance to Their Neurotoxicity: Implications for the Pathogenesis of Alzheimer's Disease. J. Bioscience and Bioengineering 99, 437-447.

Gravina, S. A., Ho, L., Eckman, C. B., Long, K. E., Otvos Jr., L., Younkin, L. H., Suzuki, N., and Younkin, S. G. (1995) Amyloid-β protein (Aβ) in Alzheimer's Disease Brain J. Biol. Chem. 27-7013-7016.

(9) Roher, A. E., Lowenson, J. D., Clarke, S., Woods, A. S., Cotter, R. J., Gowing, E., and Ball, M. J. (1993) β-amyloid-(1-42) is a major component of cerebrovascular amyloid deposits: implications for the pathology of Alzheimer disease. Proc. Natl. Acad. Sci. USA 90, 10836-40.

(10) Jarrett, J., Berger, E., and Lansbury Jr, P. (1993) The carboxy terminus of the β-amyloid protein is critical for the seeding of amyloid formation: implications for the pathogenesis of Alzheimer's disease. Biochemistry 32, 4693-7.

(11) Esler, W. P., Stimson, E. R., Ghilardi, J. R., Felix, A. M., Lu, Y., Vinters, H., Mantyh, P., and Maggio, J. (1997) Aβ deposition inhibitor screen using synthetic amyloid. Nat. Biotech. 15, 258-263.

(12) Blanchard, B. J., Chen, A., Rozeboom, L. M., Stafford, K. A., Weigele, P., and Ingram, V. M. (2004) Efficient reversal of Alzheimer's disease fibril formation and elimination of neurotoxicity by a small molecule. Proc. Nat. Acad. Sci. USA 101, 14326-14332.

(13) Kayed, R., Head, E., Thompson, J., McIntire, T., Milton, S., Cotman, C., and Glabe, C. (2003) Common Structure of Soluble Amyloid Oligomers Implies Common Mechanism of Pathogenesis Science 300, 486-489.

(14) Lambert, M., Barlow, A., Chromy, B., Edwards, C., Freed, R., Morgan, T., Rozovsky, I., Trommer, B., Viola, K., Wals, P., Zhang, C., Finch, C., Krafft, G. A., and L., K. W. (1998) Diffusible, nonfibrillar ligands derived from Aβ 1-42 are potent central nervous system neurotoxins. Proc. Nat. Acad. Sci. USA 95, 6448-6453.

(15) Whalen, B., Selkoe, D. J., and Hartley, D. (2005) Small non-fibrillar assemblies of amyloid-β protein bearing the Arctic mutation induce rapid neuritic degeneration. . Neurobiology of Disease 20, 254-266.

(16) Lesne, S., Koh, M. T., Kotilinek, L., Kayed, R., Glabe, C. G., Yang, A., Gallagher, M., and Ashe, K. H. (2006) A specific amyloid-β protein assembly in the brain impairs memory. Nature 440, 352-357.

(17) Waldo, G. S., Standish, B. M., Berendzen, J., and Terwilliger, T. C. (1999) Rapid protein-folding assay using green fluorescent protein. Nat. Biotech. 17, 691-695.

(18) Wurth, C., Guimard, N. K., and Hecht, M. H. (2002) Mutations that Reduce Aggregation of the Alzheimer's Aβ42 Peptide: an Unbiased Search for the Sequence Determinants of Aβ Amyloidogenesis. J. Mol. Biol. 319,1279-1290.

(19) Kim, W., and Hecht, M. H. (2005) Sequence Determinants of Enhanced Amyloidogenicity of Alzheimer Aβ42 peptide relative to Aβ40. J. Biol. Chem 280, 35069-35076.

(20) Coustou-Linares, V. Maddelein, M-L, Begueret, J. and Saupe, S. (2001) Mol. Microbiology 42, 1325-1335.

(21) Cubitt, A., Heim, R., Adams, S., Boyd, A., Gross, L., and Tsien, R. (1995) Understanding, improving and using green fluorescent proteins. Trends Biochem. Sci. 20, 448-455.

(22) Harper, J., and Lansbury Jr., P. (1997) Models of amyloid seeding in Alzheimer's disease and Scrapie: mechanistic truths and physiological consequences of the time-dependent solubility of amyloid proteins. Annu. Rev. Biochem. 66, 385-407.

(23) Lee, E., Leng, L., Zhang, B., Kwong, L., Trojanowski, J., Abel, T., and Lee, V. (2006) Targeting Amyloid-β peptide (Aβ) Oligomers by Passive Immunization with a Conformation-selective Monoclonal Antibody Improves Learning and Memory in Aβ Precursor Protein (APP) Transgenic Mice. J. Biol. Chem. 281, 4292-4299.

(24) Kokubo, H., Kayed, R., Glabe, C., and Yamaguchi, H. (2005) Soluble Aβ oligomer ultrastructually localize to cell process and might be related to synaptic dysfunction in Alzheimer's disease brain. Brain Res. 1031, 222-228.

(25) Tsien, R. Y. (1998) The Green Fluorescent Protein. Annu. Rev. Biochem. 67, 509-544.

(26) Lippincoft-Schwartz, J., and Patterson, G. H. (2003) Development and Use of Fluorescent Protein Markers in Living Cells. Science 300, 87-91.

(27) Pardridge, W. M. (1998) CNS Drug Design Based on Principles of Blood-Brain Barrier Transport. J. Neurochem. 70, 1781-1792.

(28) Crameri, A., Whitehom, E. A., Tate, E. and Stemmer, W. P. (1996) Improved green fluorescent protein by molecular evolution using DNA. Nat. Biotechnol. 14, 315-319.

(29) Studier, F. W., Rosenberg, A. H., Dunn, J. J., and Dubendorff, J. W. (1990) Use of T7 RNA polymerase to direct expression of cloned genes, in Methods in Enzymology (Goeddel, D., Ed.) pp 60-89, Academic Press, Orlando, Florida.

(30) Bork, J. T., Lee, J., and Chang, Y-T. (2003) Palladium-catalyzed cross-coupling reaction of resin bound chlorotriazines. Tetrahedron Lett. 44, 6141-6144.

(31) Khersonsky, S. M., Jung, D., Kang, T., Walsh, D. P., Moon, H., Jo, H., Jacobson, E., Shetty, V., Neubert, T. A., and Chang, Y-T. (2003) Facilitated Forward Chemical Genetics Using a Tagged Triazine Library and Zebrafish embryo screening. J. Am. Chem. Soc. 125, 11804-11805.

(32) Bitan, G., and Teplow, D. (2005) Preparation of Aggregate-Free, low Molecular Weight Amyloid-β for Assembly and Toxicity Assays, in Methods in Molecular Biology (Sigurdsson, E. M., Ed.) pp 3-9, Humana Press, Totowa.

(33) Levine-IlIl, H. (1993) Thioflavin T interaction with synthetic Alzheimer's disease β-amyloid peptides: Detection of amyloid aggregation in solution. Protein Sci. 2, 404-410.

(34) Jao, S., Ma, K., Talafous, J., Orlando, R., and Zagorski, M. G. (1997) Trifluoroacetic acid pretreatment reproducibly disaggregates the amyloid-β peptide. Amyloid: Int, J. Exp. Clin. Invest. 4, 240-252.

(35) Li, J., Zhu, M., Manning-Bog, A., Di Monte, D., and Fink, A. (2004) Dopamine and L-Dopa disaggregate amyloid fibrils: Implications for Parkinson's and Alzheimer's disease. FASEB 18, 962-964.

(36) Ono, K., Hasegawa, K., Naiki, H., and Yamada, M. (2004) Anti-amyloidogenic activity of tannic acid and its activity to destabilize Alzheimer's β-amyloid fibrils in vitro. Biochim. Biophys. Acta 1690,193-202.

(37) Walsh, D. M., Townsend, M., Podlisny, M. B., Shankar, G. M., Fadeeva, J. V., Agnaf, O. E., Hartley, D. M., and Selkoe, D. J. (2005) Certain Inhibitors of Synthetic Amyloid β-Peptide (Aβ) Fibrillogenesis Block Oligomerization of Natural Aβ and Thereby Rescue Long-Term Potentiation. J. Neurosci. 25, 2455-2462.

What is claimed is:

1. A screening assay for identifying inhibitors of polypeptide aggregation comprising:
a) forming a mixture of a test substance with an expression system, wherein the expression system comprises a nucleic acid encoding a fusion protein having a polypeptide domain that self-aggregates and a reporter protein domain that has an observable reporter function;
b) activating the expression system in the mixture such that the fusion protein is expressed;
c) monitoring the observable reporter function of the mixture having the test substance and comparing to the observable reporter function of the mixture in the absence of the test substance; and
d) determining from step c) whether the test substance inhibits aggregation of the polypeptide domain; wherein step a) may be performed before, during or after step b).

2. The assay of claim 1 wherein the polypeptide domain that self-aggregates comprises a polypeptide that aggregates in a disease state.

3. The assay of claim 2 wherein the polypeptide domain that self-aggregates is an Aβ polypeptide.

4. The assay of claim 1 wherein the observable reporter function is fluorescence.

5. The assay of claim 1 wherein the reporter protein domain that has an observable reporter function is a Green Fluorescent Protein (GFP) domain or a mutant form of a GFP domain.

6. The assay of claim 1 wherein the reporter protein domain that has an observable reporter function has an observable function that is activated by correct protein folding.

7. The assay of claim 1 wherein first the test substance is combined with the expression system and then the expression system is activated.

8. The assay of claim 7 wherein the expression system is activated by contacting with isopropyl-β-D-thiogalactopyranoside.

9. The assay of claim 1 wherein the expression system comprises a recombinant cell.

10. The assay of claim 9 wherein the recombinant cell is an E. coli cell.

11. The assay of claim 1 wherein the expression system comprises a cell-free transcription/translation system.

12. The assay of claim 1 wherein the fusion protein consists essentially of an amyloid Aβ42 domain covalently linked by a polypeptide linker to a GFP domain.

13. The assay of claim 1 wherein the test substance is a small molecule of 2000 Dalton or less.

14. The assay of claim 1 wherein the fusion protein further comprises a linker of between 5-30 amino acids connecting the polypeptide domain that self-aggregates and the reporter protein domain that has an observable reporter function.

15. A method for assessing a structure/activity relationship for substances that inhibit polypeptide aggregation comprising:
a) identifying a first test substance and a structurally related second test substance,
b) forming a first mixture of the first test substance with an expression system, wherein the expression system comprises a nucleic acid encoding a fusion protein having a polypeptide domain that self-aggregates and a reporter protein domain that has an observable reporter function;
c) forming a second mixture of the second test substance with the expression system;
d) activating the expression system in the first mixture and the second mixture such that the fusion proteins in the first mixture and the second mixture are expressed;
e) monitoring the observable reporter function of the first mixture and comparing to the observable reporter function of the second mixture; and
f) determining from step e) the relationship of structure to inhibition of polypeptide aggregation; wherein steps b) and c) may be performed before, during, or after step d).

16. The assay of claim 15 wherein the polypeptide domain that self-aggregates is an Aβ polypeptide.

17. The assay of claim 15 wherein the observable reporter function is fluorescence.

18. The assay of claim 15 wherein the reporter protein domain that has an observable reporter function is a Green Fluorescent Protein (GFP) domain or a mutant form of a GFP domain.

19. The assay of claim 15 wherein the expression system comprises a recombinant cell.

20. The assay of claim 15 wherein the first and second test substances differ only in one substituent of a common core structure.


SEQUENCE LISTING (2 sequences)

SEQ ID NO: 1 - protein, 12 aa, Artificial
  Synthetic sequence
  ref <301>: AUTHORS: Waldo, G.S., Standish, B. M., Berendzen, J, and Terwilliger, T.C.
  ref <302>: TITLE: Rapid protein-folding assay using green fluorescent protein
  ref <303>: JOURNAL: Nature Biotechnology
  ref <304>: VOLUME: 17
  ref <306>: PAGES: 691-695
  ref <307>: DATE: 1999-07
GSAGSAAGSGEF

SEQ ID NO: 2 - protein, 16 aa, Artificial
  Synthetic Sequence
GGGSGGGSGGGSGGGS